(19) United States
(12) Patent Application Publication
(10) Pub. No.: US 2026/0117267 A1
(43) Pub. Date: Apr. 30, 2026

(54) STREPTOMYCES STRAIN AND USE THEREOF IN SYNTHESIS OF PHOSPHATIDYLSERINE

(71) Applicants: L & P FOOD INGREDIENT CO., LTD, Shaoguan (CN); GUANGYE L & P FOOD INGREDIENT CORP., LTD., Guangzhou (CN)

(72) Inventors: Shilei HAN, Guangzhou (CN); Na WEI, Guangzhou (CN); Jie LIN, Shaoguan (CN); Rihui CHE, Shaoguan (CN)

(73) Assignees: L & P FOOD INGREDIENT CO., LTD, Shaoguan (CN); GUANGYE L & P FOOD INGREDIENT CORP., LTD., Guangzhou (CN)

(21) Appl. No.: 19/415,720

(22) Filed: Dec. 10, 2025

Related U.S. Application Data
(63) Continuation of application No. PCT/CN2024/131157, filed on Nov. 10, 2024.

(30) Foreign Application Priority Data
Oct. 29, 2024 (CN) 202411523106.X

(51) Int. Cl. C12P 7/6481; C12N 1/20

(52) U.S. Cl. CPC C12P 7/6481; C12N 1/20

(57) ABSTRACT

Provided is a Streptomyces and its use in synthesis of phosphatidylserine. A fermentation broth produced by the Streptomyces, using soybean lecithin and L-serine as raw materials, can simultaneously convert phosphatidylcholine (PC), phosphatidylethanolamine (PE), and phosphatidylinositol (PI) in lecithin to efficiently produce phosphatidylserine (PS). The Streptomyces, designated as Streptomyces sp. LN2, is deposited at China General Microbiological Culture Collection Center (CGMCC) on Apr. 1, 2024, with an accession number CGMCC NO. 30230.

### CROSS-REFERENCE TO RELATED APPLICATIONS

This application is a Continuation of International Application No. PCT/CN2024/131157, filed on Nov. 10, 2024, which claims priority to Chinese Patent Application No. 202411523106.X, filed on Oct. 29, 2024, the entire contents of each of which are hereby incorporated by reference.

### SEQUENCE LISTING

The instant application contains a Sequence Listing which is submitted electronically in XML format and is hereby incorporated by reference in its entirety. The XML copy, created on Dec. 5, 2025, is named “2025-12-05-Sequence List-6D801-H001US00,” and is 3,396 bytes in size.

### TECHNICAL FIELD

The present disclosure relates to the field of biotechnology, and in particular to a Streptomyces and its use in synthesis of phosphatidylserine.

### BACKGROUND

Phospholipids are commonly referred to as lecithin. In a narrow sense, phospholipids specifically refer to phosphatidylcholine (PC). In a broad sense, phospholipids refer to a general term for a class of mixtures containing phosphatidyl glycerides. Based on different phosphatidyl groups contained in the phospholipids, the phospholipids can be classified into phosphatidylethanolamine (PE), phosphatidylinositol (PI), phosphatidylserine (PS), phosphatidylcholine (PC), phosphatidylglycerol (PG), phosphatidic acid (PA), etc. PS is an only phospholipid that can regulate a state of key proteins in a cell membrane, which plays an important role in maintaining a structure and function of the cell membrane. PS can also activate metabolic synthesis of a plurality of enzymes. Due to strong lipophilicity, PS can rapidly pass through a blood-brain barrier and enter the brain after absorption, helping to relax vascular smooth muscle cells and increase cerebral blood supply. PS is known as a major emerging “smart nutrient” following choline-based “brain gold” (docosahexaenoic acid, DHA). Research indicates that PS has effects of preventing Alzheimer's disease, improving brain memory, alleviating depression, improving children's attention, etc. PS has been listed as a new resource food and a functional food, leading to a wide application in health products, cosmetics, and pharmaceutical industries. However, natural abundance of PS is low, and an extraction process is complicated, making production unable to meet market demand. Therefore, preparing a high-purity and high-quality PS product is of significant importance.

Subsequent research found that PS, PC, and PE share similar structures and can undergo interconversion. Therefore, research on an enzymatic preparation of functional phospholipids has been carried out successively. An enzymatic catalytic manner for preparing PS has characteristics of high product quality, simple operation, and a mild process condition. Therefore, a manner of bio-enzymatic catalysis for synthesizing the functional phospholipids is highly valuable and more readily yields products that are difficult to obtain through chemical catalysis.

Currently, a bio-enzymatic production of PS mainly uses phospholipase D (PLD) to catalyze a transphosphatidylation reaction between the lecithin (derived from a soybean, a sunflower seed, an egg, etc.) and L-serine to produce PS. PLD is ubiquitous in plants, animals, and microorganisms. Microbial-derived PLD has become a most widely used enzyme due to high catalytic performance and a simple purification process of the microbial-derived PLD. Reported PLD-producing microorganisms mainly include Streptomyces, Coryne bacterium, Escherichia coli, Salmonella, Pseudomonad, Vibrio harveyi, Bacillus cereus, Achromobacter, and Aspergillus oryzae. The reported PLD-producing microorganisms mostly use the lecithin as a substrate. However, in addition to PC as a main component, soybean lecithin contains several important components, i.e., PE (about 15%) and PI (about 15%). Nevertheless, these microorganisms can only catalyze a transphosphatidylation reaction of PC to produce PS, making it difficult to obtain a high-content PS from lecithin with a low-content PC. This leads to a significant decrease in a utilization rate of the raw material lecithin, thereby increasing production costs. Even if PC is directly used as a raw material to synthesize PS, a reaction cost still increases because PC is expensive.

Therefore, it is necessary to screen a strain that can use the lecithin as the raw material and can simultaneously convert both PC and PE into PS and provide a safe, environmentally friendly, and low-cost PS production method with a high raw material utilization rate, which is of significant importance for an industrial preparation of PS.

### SUMMARY

One or more embodiments of the present disclosure provide a method for synthesizing phosphatidylserine, comprising: a) homogenously mixing soybean lecithin, L-serine, and a fermentation broth of Streptomyces sp. LN2, deposited at China General Microbiological Culture Collection Center (CGMCC), with an accession number CGMCC NO. 30230, to form a reaction system; b) subjecting the reaction system to an oscillating reaction; c) after completion of the oscillating reaction, adding water to the reaction system to dissolve the L-serine, followed by centrifugation to obtain a precipitate; and d) subjecting the precipitate to vacuum freeze-drying to obtain a phosphatidylserine product.

In some embodiments, the oscillating reaction in step b) is performed by using the fermentation broth of Streptomyces sp. LN2 as a catalyst and the soybean lecithin and the L-serine as raw materials, thereby simultaneously catalyzing synthesis of phosphatidylserine from phosphatidylcholine, phosphatidylethanolamine, and phosphatidylinositol in the raw materials.

In some embodiments, a reaction temperature of the oscillating reaction is within a range of 40° C.-45° C., and a reaction time of the oscillating reaction is within a range of 6-8 hours.

In some embodiments, steps c) and d) comprise: after completion of the oscillating reaction, adding water to the reaction system to dissolve the L-serine, followed by centrifugation to obtain a first aqueous phase and a first precipitate; washing the first precipitate with water, followed by centrifugation to obtain a second aqueous phase and a second precipitate; combining the first aqueous phase and the second aqueous phase, and concentrating via membrane filtration to recover the L-serine; and subjecting the second precipitate to vacuum freeze-drying to obtain the phosphatidylserine product.

### BRIEF DESCRIPTION OF THE DRAWINGS

The present disclosure will be further illustrated by way of exemplary embodiments, which will be described in detail with reference to the drawings. These embodiments are non-limiting, wherein:

FIG. 1 is a colony morphology diagram of Streptomyces sp. LN2 cultured on solid medium according to some embodiments of the present disclosure;

FIG. 2 is a phylogenetic tree diagram of Streptomyces sp. LN2 according to some embodiments of the present disclosure;

FIG. 3 is a schematic diagram illustrating a liquid chromatography detection result of raw materials for a phosphatidylserine synthesis reaction using Streptomyces sp. LN2 according to some embodiments of the present disclosure;

FIG. 4 is a schematic diagram illustrating a liquid chromatography detection result after completion of a phosphatidylserine synthesis reaction using Streptomyces sp. LN2 according to some embodiments of the present disclosure.

### BIOLOGICAL MATERIAL DEPOSIT INFORMATION

Streptomyces sp. LN2 was deposited at the China General Microbiological Culture Collection Center (CGMCC), with the deposit address at No. 3, Yard 1, Beichen West Road, Chaoyang District, Beijing, China. A deposit date is Apr. 1, 2024, and an accession number is CGMCC No. 30230.

### DETAILED DESCRIPTION

To make the objectives, technical solutions, and advantages of the embodiments of the present disclosure clearer, the technical solutions in the embodiments of the present disclosure will be clearly and completely described below in combination with the embodiments of the present disclosure. Obviously, the described embodiments are a part of the embodiments of the present disclosure, and not all of the embodiments. Based on the embodiments in the present disclosure, all other embodiments obtained by those of ordinary skill in the art without making creative efforts fall within the scope of protection of the present disclosure.

One or more embodiments of the present disclosure provide a phosphatidylserine (PS)-producing strain screened from a soil sample, i.e., a Streptomyces sp. LN2, which was deposited at the China General Microbiological Culture Collection Center (CGMCC) on Apr. 1, 2024, Institute of Microbiology, Chinese Academy of Sciences, No. 3, Yard 1, Beichen West Road, Chaoyang District, Beijing, with an accession number CGMCC No. 30230.

One or more embodiments of the present disclosure provide a use of the Streptomyces sp. LN2 in synthesis of phosphatidylserine.

One or more embodiments of the present disclosure provide a method for synthesizing phosphatidylserine, comprising: a) homogenously mixing soybean lecithin, L-serine, and a fermentation broth of Streptomyces sp. LN2, deposited at CGMCC, with an accession number CGMCC NO. 30230, to form a reaction system; b) subjecting the reaction system to an oscillating reaction; c) after completion of the oscillating reaction, adding water to the reaction system to dissolve the L-serine, followed by centrifugation to obtain a precipitate; and d) subjecting the precipitate to vacuum freeze-drying to obtain a phosphatidylserine product.

In some embodiments, the oscillating reaction in step b) is performed by using the fermentation broth of Streptomyces sp. LN2 as a catalyst and the soybean lecithin and the L-serine as raw materials, thereby simultaneously catalyzing synthesis of phosphatidylserine from phosphatidylcholine, phosphatidylethanolamine, and phosphatidylinositol in the raw materials.

In some embodiments, a reaction temperature of the oscillating reaction is within a range of 40-45° C., and a reaction time of the oscillating reaction is within a range of 6-8 hours (h).

In some embodiments, the reaction temperature of the oscillating reaction may be 40° C., 41° C., 42° C., 43° C., 44° C., or 45° C., and the reaction time of the oscillating reaction may be 6 h, 6.5 h, 7 h, 7.5 h, or 8 h.

In some embodiments, steps c) and d) comprise: after completion of the oscillating reaction, adding water to the reaction system to dissolve the L-serine, followed by centrifugation to obtain a first aqueous phase and a first precipitate; washing the first precipitate with water, followed by centrifugation to obtain a second aqueous phase and a second precipitate; combining the first aqueous phase and the second aqueous phase, and concentrating via membrane filtration to recover the L-serine; and subjecting the second precipitate to vacuum freeze-drying to obtain the phosphatidylserine product.

In the embodiments of the present disclosure, by using the fermentation broth of the screened Streptomyces as the catalyst, and using the soybean lecithin and the L-serine as the raw materials, the phosphatidylcholine (PC), the phosphatidylethanolamine (PE), and the phosphatidylinositol (PI) in lecithin can be simultaneously converted to efficiently produce PS, thereby improving a raw material utilization rate.

Technical solutions of the present disclosure are described in detail below in combination with specific examples, so that those skilled in the art can better understand and implement the technical solutions of the present disclosure. Reagents or materials used in the examples, unless otherwise specified, are all from commercial sources.

### Example 1 Determination for Transphosphatidylation Activity of Crude Fermentation Enzyme Broth

### (1) Determination by Catalyzing the Conversion of PC and L-Serine to PS

An appropriate amount of soy-derived PC was dissolved in 10 mL of chromatographical grade chloroform (a final concentration of 5 mg/mL) to form an organic phase; another 10 mL of crude fermentation enzyme broth was mixed with L-serine (a final concentration of 50 mg/mL), Triton X-100 (v/v, 2.4%), and CaCl₂ (a final concentration of 80 mM) to form an aqueous phase; the organic phase and the aqueous phase were mixed at a ratio of 1:1 in an Erlenmeyer flask, which was placed on a constant temperature oscillating shaker and subjected to an oscillating reaction at 37° C. for 1-8 h.

### (2) Liquid Chromatography Detection

After the reaction ended, 500 μL of a reaction solution sample was first taken and centrifuged at 12000 rpm for 10 minutes (min); an upper organic phase was collected, after evaporating the organic phase, the residue was redissolved in mobile phase A (n-hexane-isopropanol-acetic acid-triethylamine, with a volume ratio of 820:170:10:0.8); the redissolved solution was passed through a 0.45 m organic membrane, and analyzed by high performance liquid chromatography (HPLC).

Chromatographic conditions: lichrosphere 100 diol column (4 mmx 125 mm); evaporative light scattering detector (ELSD); mobile phase: mobile phase A (n-hexane-isopropanol-acetic acid-triethylamine, with a volume ratio of 820:170:10:0.8), mobile phase B (isopropanol-water-acetic acid-triethylamine, with a volume ratio of 850:140:10:0.8); flow rate: 1 mL/min; column temperature: 30° C.; injection volume: 10 μL; evaporation temperature: 80° C.; nebulization temperature: 50° C.; nitrogen gas flow rate: 1 L/min; gradient elution: performed according to Table 1.

Enzyme activity was defined as: an amount of enzyme required to catalyze the conversion of PC into 1 μmol of phosphatidylserine per minute under a condition of 37° C.

TABLE 1
Gradient elution program

| | Running | Mobile | Mobile |
|---|---|---|---|
| Step | Time/min | Phase A/(%) | Phase B/(%) |
| 1 | 0 | 100 | 0 |
| 2 | 20 | 50 | 50 |
| 3 | 25 | 0 | 100 |
| 4 | 29 | 0 | 100 |
| 5 | 30 | 100 | 0 |
| 6 | 35 | 100 | 0 |

### Example 2 Screening and Identification of Streptomyces sp. LN2

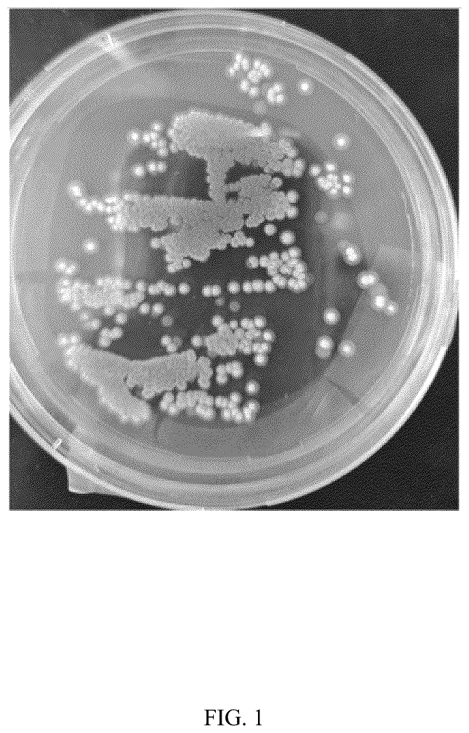

According to Bergey's Manual of Determinative Bacteriology, the strain was first identified as Streptomyces through morphological observation and physiological and biochemical tests. The Streptomyces sp. LN2 of the present disclosure exhibits typical characteristics of the Streptomyces: during solid culture, hyphae are branched and aseptate, the strain is Gram-positive, and the hyphae interweave to form small and dense colonies that are dry, opaque, and difficult to pick. As shown in FIG. 1, a colony diameter of the strain reaches 3-4 mm after 5-7 days; the hyphae are initially white and later develop a light gray and velvety appearance.

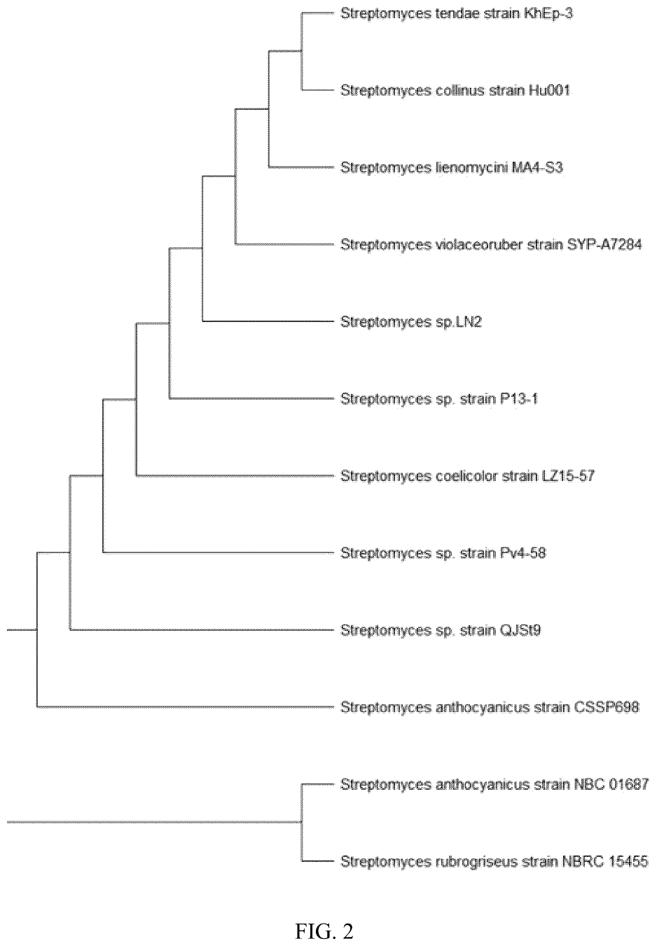

Subsequently, 16S rDNA cluster analysis was performed for identification. 16S rDNA sequence of the isolated strain was sequenced by General Biosystems (Anhui) Co., Ltd. The 16S rDNA sequences of reported Streptomyces were downloaded from National Center for Biotechnology Information (NCBI) database. A phylogenetic tree of 16S rDNA of the strain was obtained through analysis and comparison using software MEGA 10.0. The results, shown in FIG. 2, indicate that physiological and biochemical characteristics of the strain LN2 are consistent with those of the Streptomyces, belonging to a Streptomyces genus.

The determined 16S rDNA sequence (SEQ ID NO.1) is:

GTCGAACGATGAACCACTTCGGTGGGGATTAGTGGCGAACGGGTGA
GTAACACGTGGGCAATCTGCCCTTCACTCTGGGACAAGCCCTGGA
AACGGGGTCTAATACCGGATACTGACCCTCGCAGGCATCTGCGAG
GTTCGAAAGCTCCGGCGGTGAAGGATGAGCCCGCGGCCTATCAGC
TTGTTGGTGAGGTAATGGCTCACCAAGGCGACGACGGGTAGCCGG
CCTGAGAGGGCGACCGGCCACACTGGGACTGAGACACGGCCCAGA
CTCCTACGGGAGGCAGCAGTGGGGAATATTGCACAATGGGCGAAA
GCCTGATGCAGCGACGCCGCGTGAGGGATGACGGCCTTCGGGTTG
TAAACCTCTTTCAGCAGGGAAGAAGCGAAAGTGACGGTACCTGCA
GAAGAAGCGCCGGCTAACTACGTGCCAGCAGCCGCGGTAATACGT
AGGGCGCAAGCGTTGTCCGGAATTATTGGGCGTAAAGAGCTCGTA
GGCGGCTTGTCACGTCGGTTGTGAAAGCCCGGGGCTTAACCCCGG
GTCTGCAGTCGATACGGGCAGGCTAGAGTTCGGTAGGGGAGATCG
GAATTCCTGGTGTAGCGGTGAAATGCGCAGATATCAGGAGGAACA
CCGGTGGCGAAGGCGGATCTCTGGGCCGATACTGACGCTGAGGAG
CGAAAGCGTGGGGAGCGAACAGGATTAGATACCCTGGTAGTCCAC
GCCGTAAACGGTGGGCACTAGGTGTGGGCAACATTCCACGTTGTC
CGTGCCGCAGCTAACGCATTAAGTGCCCCGCCTGGGGAGTACGGC
CGCAAGGCTAAAACTCAAAGGAATTGACGGGGGCCCGCACAAGCG
GCGGAGCATGTGGCTTAATTCGACGCAACGCGAAGAACCTTACCA
AGGCTTGACATACACCGGAAAGCATCAGAGATGGTGCCCCCCTTG
TGGTCGGTGTACAGGTGGTGCATGGCTGTCGTCAGCTCGTGTCGT
GAGATGTTGGGTTAAGTCCCGCAACGAGCGCAACCCTTGTCCCGT
GTTGCCAGCAAGCCCTTCGGGGTGTTGGGGACTCACGGGAGACCG
CCGGGGTCAACTCGGAGGAAGGTGGGGACGACGTCAAGTCATCAT
GCCCCTTATGTCTTGGGCTGCACACGTGCTACAATGGCCGGTACA
ATGAGCTGCGATACCGCAAGGTGGAGCGAATCTCAAAAAGCCGGT
CTCAGTTCGGATTGGGGTCTGCAACTCGACCCCATGAAGTCGGAG
TCGCTAGTAATCGCAGATCAGCATTGCTGCGGTGAATACGTTCCC
GGGCCTTGTACACACCGCCCGTCACGTCACGAAAGTCGGTAACAC
CCGAAGCCG.

Specific screening steps for the strain in the present disclosure were as follows:

(1) Primary screening on plates: the collected soil samples were processed and incubated at a constant temperature of 28° C. using lecithin as both a substrate and an inducer, with bromocresol purple as a colorimetric indicator. Colonies capable of producing a white transparent zone around the colonies were identified as lecithin-converting strains. Single colonies with the transparent zone were selected and cultured on a solid medium for 72 h before proceeding to secondary screening.

The used solid medium contained: yeast extract 4 g/L, maltodextrin 10 g/L, glucose 4 g/L, and agar 15 g/L, sterilized at 115° C. for 20 min.

(2) Secondary Screening in shake flasks: the strains obtained from the primary screening on plates were inoculated into a seed medium for shake flask culture. A culture temperature was 28° C., and a culture time was 5 days, resulting in a seed culture solution. The used seed medium contained: soybean peptone 20 g/L, maltodextrin 20 g/L, glucose 4 g/L, K₂HPO₄ 1 g/L, MgSO₄·7H₂O 1 g/L, CaCl₂) 0.1 g/L, and Na₂HPO₄·12H₂O 6 g/L, sterilized at 115° C. for 20 min.

(3) Fermentation and enzyme activity detection: the obtained seed culture solution was then inoculated at 2% (v/v) into a basal fermentation medium and cultured at 28° C. for 48 h to obtain a fermentation broth. The used basal fermentation medium contained: soybean peptone 30 g/L, maltodextrin 20 g/L, glucose 7 g/L, K₂HPO₄ 1 g/L, MgSO₄·7H₂O 1 g/L, CaCl₂ 0.1 g/L, and Na₂HPO₄·12H₂O 6 g/L, sterilized at 115° C. for 20 min. The fermentation broth was centrifuged at 6000 rpm for 30 min to remove Streptomyces sp. LN2 cells, and the resulting supernatant was further ultrafiltered through an ultrafiltration membrane to remove salts and miscellaneous proteins, thereby obtaining a crude enzyme broth. Enzyme activity was determined according to the above enzyme activity detection method, and strains with high transphosphatidylation activity were screened for preparation of the phosphatidylserine. As a result, a strain with a highest transphosphatidylation activity of up to 58.4 U/mL was screened and named the Streptomyces sp. LN2.

### Example 3 Strain Fermentation

The Streptomyces sp. LN2 screened in the above Example 2 was subjected to fermentation and scale-up culture under the following conditions.

(1) Plate culture: a plate medium was prepared by using soybean peptone 20 g/L, maltodextrin 20 g/L, glucose 4 g/L, K₂HPO₄ 1 g/L, MgSO₄·7H₂O 1 g/L, CaCl₂ 0.1 g/L, Na₂HPO₄·12H₂O 6 g/L, and agar 15 g/L, followed by sterilization at 115° C. for 20 min. The Streptomyces sp. LN2 was inoculated onto the plate medium and cultured at 28° C. for 72 h.

(2) Primary seed solution culture: a 250 mL Erlenmeyer flask containing 6 glass beads was filled with 60 mL of medium that was prepared by using soybean peptone 20 g/L, maltodextrin 20 g/L, glucose 4 g/L, K₂HPO₄ 1 g/L, MgSO₄·7H₂O 1 g/L, CaCl₂ 0.1 g/L, and Na₂HPO₄·12H₂O 6 g/L and sterilizing at 115° C. for 20 min. Several large colonies of the Streptomyces sp. LN2 obtained in step (1) were picked and inoculated into the primary seed medium, and cultured at 200 rpm and 28° C. for 48 h.

(3) Secondary seed solution culture: a 2 L Erlenmeyer flask containing 8 glass beads was filled with a 300 mL of medium that was prepared by using soybean peptone 30 g/L, maltodextrin 20 g/L, glucose 4 g/L, K₂HPO₄ 1 g/L, MgSO₄·7H₂O 1 g/L, CaCl₂ 0.1 g/L, and Na₂HPO₄·12H₂O 6 g/L and sterilizing at 115° C. for 20 min. The entire primary seed culture solution obtained in step (2) was inoculated, and cultured at 200 rpm and 28° C. for 48 h.

(4) Fermentation broth culture: a 5 L fermenter was filled with a 3 L of fermentation medium that was prepared by using soybean peptone 30 g/L, maltodextrin 20 g/L, glucose 7 g/L, K₂HPO₄ 1 g/L, MgSO₄·7H₂O 1 g/L, CaCl₂) 0.1 g/L, Na₂HPO₄·12H₂O 6 g/L, and antifoam agent 1 mL/L and sterilizing at 115° C. for 20 min. The entire secondary seed culture solution obtained in step (3) was inoculated into the fermentation medium. An initial stirring speed was 200 rpm with an aeration rate of 1 vvm, and cultivation proceeded at 28° C. for 48 h to obtain the fermentation broth.

(5) Centrifugation and ultrafiltration to obtain crude enzyme broth: the fermentation broth obtained in step (4) was allowed to stand for a period of time, then centrifuged at 6000 rpm for 30 min to remove Streptomyces sp. LN2 cells. The resulting supernatant was further ultrafiltered through an ultrafiltration membrane to remove salts and miscellaneous proteins, thereby obtaining the crude enzyme broth.

### Example 4 Preparation of Phosphatidylserine

An appropriate amount of the crude fermentation enzyme broth from the above Example 3 was taken to prepare 270 mL of an enzyme-containing aqueous solution with an enzyme activity of 10 U/mL, and a pH was adjusted to 7.0. 50 g of soybean lecithin raw material (with a PC content of 44.33%) and 40 g of L-serine were added to the enzyme-containing aqueous solution, homogenously mixed and stirred, and subjected to an oscillating reaction at 40-45° C. for 6-8 h.

After the reaction, 500 μL of the reaction solution sample was first taken and centrifuged at 12000 rpm for 10 min. The upper organic phase was collected, after evaporating the organic solvent, the residue was redissolved in mobile phase A (n-hexane-isopropanol-acetic acid-triethylamine, with a volume ratio of 820:170:10:0.8). The resulting solution was then filtered through a 0.45 m organic membrane and analyzed by HPLC.

Subsequently, 270 mL of water was added to the reaction system to dissolve the L-serine with stirring. An aqueous phase was separated by centrifugation to obtain 90 g of a centrifugal precipitate. The centrifugal precipitate was washed with 450 mL of pure water, and an aqueous phase was separated by centrifugation. The aqueous phases obtained from the two centrifugal separations (totaling about 990 mL) were concentrated to 150 mL by membrane (e.g., a reverse osmosis membrane) filtration to obtain an L-serine aqueous solution for a next batch of reaction, and 90 g of a secondary centrifugal precipitate was obtained. The precipitates obtained from the two centrifugations were vacuum freeze-dried to obtain 43 g of a product containing high-purity phosphatidylserine.

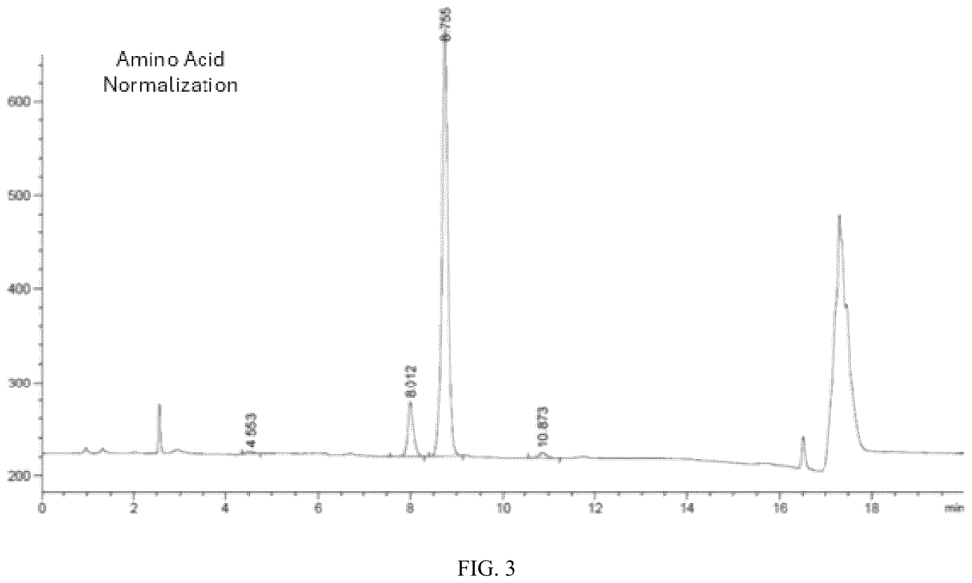

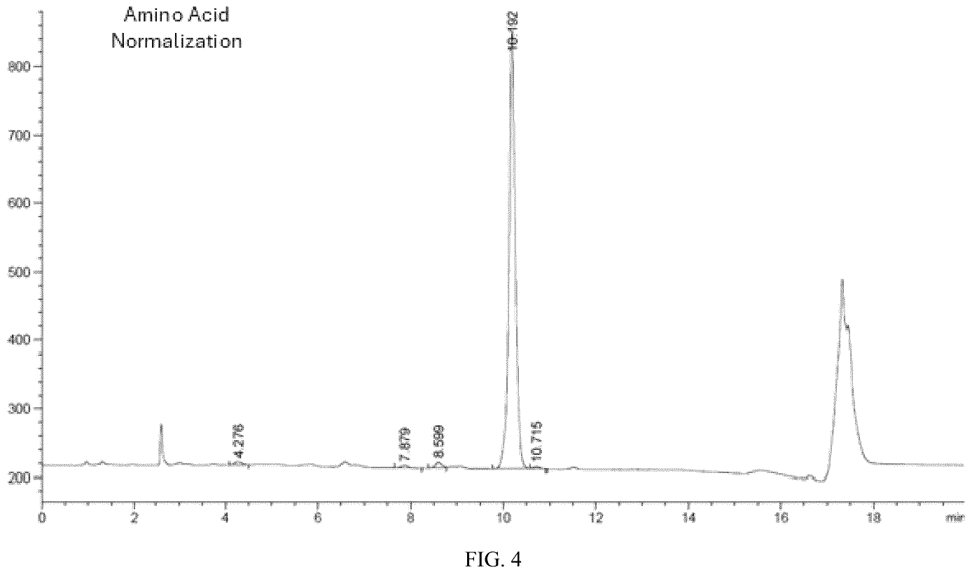

The liquid phase detection results for the soybean lecithin raw material and the post-reaction mixture are shown in FIGS. 3 and 4. As shown in FIG. 3, substances detected in the raw materials, in order of retention time, are: PA at 4.553 min, PE at 8.012 min, PC at 8.755 min, and PI at 10.873 min. As shown in FIG. 4, substances detected after the reaction, in order of the retention time, are: PA at 4.276 min, PE at 7.879 min, PC at 8.599 min, a new peak (i.e., a PS product) at 10.192 min, and PI at 10.715 min. A percentage content of each component in reactants is shown in Table 2. The results indicate that the Streptomyces sp. LN2 of the present disclosure not only converts PC, a main component in the lecithin, to PS, but also significantly reduces contents of PE and PI. This indicates that the Streptomyces sp. LN2 also possesses an activity to convert PE and PI into PS, allowing full utilization of the raw material, and a PC conversion rate is as high as 109.77%.

TABLE 2
Content of each component in reactants

| Content of each phospholipid | | | | | Content of each phospholipid | | | | | PC |
|---|---|---|---|---|---|---|---|---|---|---|
| component in raw material (%) | | | | | component in reaction product (%) | | | | | conversion |
| PA | PE | PC | PS | PI | PA | PE | PC | PS | PI | rate (%) |
| 2.00 | 11.38 | 44.33 | 0 | 5.74 | 2.47 | 2.04 | 3.93 | 61.38 | 2.59 | 109.77 |

Note:
PC conversion rate = (PS content in reaction product ÷ PC content in raw material) × 100%.

### Example 5 Verification of Activity of Streptomyces sp. LN2 for Converting PE to PS

An appropriate amount of PE was dissolved in 10 mL of chromatographical grade chloroform (a final concentration of 5 mg/mL) to form an organic phase. Separately, 10 mL of a crude fermentation enzyme broth was mixed with L-serine (a final concentration of 50 mg/mL), Triton X-100 (v/v, 2.4%), and CaCl₂ (a final concentration of 80 mM) to form an aqueous phase. The organic phase and the aqueous phase were mixed at a ratio of 1:1 in an Erlenmeyer flask, which was placed on a constant-temperature oscillating shaker and subjected to an oscillating reaction at 37° C. for 1-8 h.

Enzyme activity was defined as an amount of enzyme required to catalyze the conversion of PE into 1 μmol of phosphatidylserine per minute under a condition of 37° C.

A content of phosphatidylserine was determined according to the sample processing method and HPLC-ELSD detection method in Example 1, and an enzyme activity was calculated. An activity of the Streptomyces sp. LN2 for converting PE to PS reaches 33.7 U/mL.

### Example 6 Verification of Activity of Streptomyces sp. LN2 for Converting PI to PS

An appropriate amount of PI was dissolved in 10 mL of chromatographical grade chloroform (a final concentration of 5 mg/mL) to form an organic phase. Separately, 10 mL of a crude fermentation enzyme broth was mixed with L-serine (a final concentration of 50 mg/mL), Triton X-100 (v/v, 2.4%), and CaCl₂ (a final concentration of 80 mM) to form an aqueous phase. The organic phase and the aqueous phase were mixed at a ratio of 1:1 in an Erlenmeyer flask, which was placed on a constant-temperature oscillating shaker and subjected to an oscillating reaction at 37° C. for 1-8 h.

Enzyme activity was defined as an amount of enzyme required to catalyze the conversion of PI into 1 μmol of phosphatidylserine per minute under a condition of 37° C.

A content of phosphatidylserine was determined according to the sample processing method and HPLC-ELSD detection method in Example 1, and an enzyme activity was calculated. An activity of the Streptomyces sp. LN2 for converting PI to PS reaches 12.6 U/mL.

The basic concepts have been described above. Clearly, the above detailed disclosure is merely by way of example to those skilled in the art and does not constitute a limitation to the present disclosure. Although not explicitly stated herein, those skilled in the art may make various modifications, improvements, and amendments to the present disclosure. Such modifications, improvements, and amendments are suggested in the present disclosure. Therefore, such the modifications, improvements, and amendments still fall within the spirit and scope of the exemplary embodiments of the present disclosure.

Meanwhile, the present disclosure uses specific words to describe the embodiments of the present disclosure. For example, “an embodiment,” “one embodiment,” and/or “some embodiments” mean that a certain feature, structure, or characteristic is related to at least one embodiment of the present disclosure. Therefore, it should be emphasized and noted that the references to “an embodiment” or “one embodiment” or “an alternative embodiment” two or more times in different places in the present disclosure do not necessarily refer to the same embodiment. In addition, certain features, structures, or characteristics in one or more embodiments of the present disclosure may be appropriately combined.

In addition, unless explicitly stated in the claims, the order of processing elements and sequences, the use of numbers and letters, or the use of other names in the present disclosure are not used to limit the order of the processes and methods of the present disclosure. Although the foregoing disclosure discusses some embodiments of the invention currently considered useful through various examples, it should be understood that such details are for illustrative purposes only. The appended claims are not limited to the disclosed embodiments. Instead, the claims are intended to cover all modifications and equivalent combinations that conform to the substance and scope of the embodiments of the present disclosure.

Similarly, it should be noted that, in order to simplify the expression disclosed in the present disclosure and thereby help the understanding of one or more embodiments of the invention, multiple features are sometimes grouped into one embodiment, drawing, or description thereof in the foregoing description of the embodiments of the present disclosure. However, this manner of disclosure does not mean that the object of the present disclosure requires more features than those mentioned in the claims. Rather, claimed subject matter may lie in less than all features of a single foregoing disclosed embodiment.

In some embodiments, numbers describing the quantity of ingredients and attributes are used. It should be understood that such numbers used in the description of the embodiments are modified by the modifiers “about”, “approximately”, or “substantially” in some embodiments. Unless otherwise stated, “about”, “approximately”, or “substantially” indicates that the stated number allows a variation of 20%. Accordingly, in some embodiments, the numerical parameters used in the present disclosure and claims are approximate values. These approximate values may vary according to the characteristics required by individual embodiments. In some embodiments, numerical parameters should consider the specified number of significant digits and adopt the manner of general digit retention. Although the numerical ranges and parameters used to confirm the breadth of their scope in some embodiments of the present disclosure are approximate values, such numerical values are set as precisely as possible within a feasible range in specific embodiments.

For each patent, patent application, patent application publication, and other material, such as articles, books, specifications, publications, documents, etc., cited in the present disclosure, the entire content thereof is hereby incorporated by reference into the present disclosure. Excluded are application history documents that are inconsistent with or conflict with the content of the present disclosure. Also excluded are documents that limit the broadest scope of the claims of the present disclosure (whether currently or subsequently appended to the present disclosure). It should be noted that if the description, definition, and/or use of terms in the ancillary materials of the present disclosure are inconsistent with or conflict with the content described in the present disclosure, the description, definition, and/or use of terms in the present disclosure shall prevail.

Finally, it should be understood that the embodiments described in the present disclosure are only used to illustrate the principles of the embodiments of the present disclosure. Other variations may also fall within the scope of the present disclosure. Therefore, by way of example and not limitation, alternative configurations of the embodiments of the present disclosure may be considered consistent with the teachings of the present disclosure. Accordingly, the embodiments of the present disclosure are not limited to the embodiments explicitly introduced and described in the present disclosure.

What is claimed is:

1. A method for synthesizing phosphatidylserine, comprising:
a) homogenously mixing soybean lecithin, L-serine, and a fermentation broth of Streptomyces sp. LN2, deposited at China General Microbiological Culture Collection Center (CGMCC), with an accession number CGMCC NO. 30230, to form a reaction system;
b) subjecting the reaction system to an oscillating reaction;
c) after completion of the oscillating reaction, adding water to the reaction system to dissolve the L-serine, followed by centrifugation to obtain a precipitate; and
d) subjecting the precipitate to vacuum freeze-drying to obtain a phosphatidylserine product.

2. The method according to claim 1, wherein the oscillating reaction in step b) is performed by using the fermentation broth of Streptomyces sp. LN2 as a catalyst and the soybean lecithin and the L-serine as raw materials, thereby simultaneously catalyzing synthesis of phosphatidylserine from phosphatidylcholine, phosphatidylethanolamine, and phosphatidylinositol in the raw materials.

3. The method according to claim 2, wherein a reaction temperature of the oscillating reaction is within a range of 40° C.-45° C., and a reaction time of the oscillating reaction is within a range of 6-8 hours.

4. The method according to claim 2, wherein steps c) and d) comprise:
after completion of the oscillating reaction, adding the water to the reaction system to dissolve the L-serine, followed by centrifugation to obtain a first aqueous phase and a first precipitate;
washing the first precipitate with water, followed by centrifugation to obtain a second aqueous phase and a second precipitate;
combining the first aqueous phase and the second aqueous phase, and concentrating via membrane filtration to recover the L-serine; and
subjecting the second precipitate to the vacuum freeze-drying to obtain the phosphatidylserine product.


SEQUENCE LISTING (1 sequence)

SEQ ID NO: 1 - DNA, 1360 bp, synthetic construct
  source 1..1360: mol_type = other DNA
gtcgaacgatgaaccacttcggtggggattagtggcgaacgggtgagtaacacgtgggcaatctgcccttcactctgggacaagccctggaaacggggtctaataccggatactgaccctcgcaggcatctgcgaggttcgaaagctccggcggtgaaggatgagcccgcggcctatcagcttgttggtgaggtaatggctcaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcggttgtgaaagcccggggcttaaccccgggtctgcagtcgatacgggcaggctagagttcggtaggggagatcggaattcctggtgtagcggtgaaatgcgcagatatcaggaggaacaccggtggcgaaggcggatctctgggccgatactgacgctgaggagcgaaagcgtggggagcgaacaggattagataccctggtagtccacgccgtaaacggtgggcactaggtgtgggcaacattccacgttgtccgtgccgcagctaacgcattaagtgccccgcctggggagtacggccgcaaggctaaaactcaaaggaattgacgggggcccgcacaagcggcggagcatgtggcttaattcgacgcaacgcgaagaaccttaccaaggcttgacatacaccggaaagcatcagagatggtgccccccttgtggtcggtgtacaggtggtgcatggctgtcgtcagctcgtgtcgtgagatgttgggttaagtcccgcaacgagcgcaacccttgtcccgtgttgccagcaagcccttcggggtgttggggactcacgggagaccgccggggtcaactcggaggaaggtggggacgacgtcaagtcatcatgccccttatgtcttgggctgcacacgtgctacaatggccggtacaatgagctgcgataccgcaaggtggagcgaatctcaaaaagccggtctcagttcggattggggtctgcaactcgaccccatgaagtcggagtcgctagtaatcgcagatcagcattgctgcggtgaatacgttcccgggccttgtacacaccgcccgtcacgtcacgaaagtcggtaacacccgaagccg